(19) United States
(12) Patent Application Publication
(10) Pub. No.: US 2025/0017780 A1
(43) Pub. Date: Jan. 16, 2025

(54) METHOD FOR DETERMINING A RESULT OF A POST-OPERATIVE SUBJECTIVE REFRACTION MEASUREMENT

(71) Applicant: Carl Zeiss Meditec AG, Jena (DE)

(72) Inventors: Mhd Hasan Sarhan, Munich (DE); Hessam Roodaki, Munich (DE); Abouzar Eslami, Munich (DE)

(73) Assignee: Carl Zeiss Meditec AG, Jena (DE)

(21) Appl. No.: 18/714,389

(22) PCT Filed: Nov. 29, 2022
(86) PCT No.: PCT/EP2022/083707

§ 371 Date: May 29, 2024

(30) Foreign Application Priority Data
Nov. 30, 2021 (DE) 10 2021 213 511.0

(51) Int. Cl. A61F 9/008

(52) U.S. Cl. CPC A61F 9/00804

(57) ABSTRACT

A method includes receiving, at a data processing device, an outcome of a subjective refraction measurement of at least one eye of a patient performed preoperatively, receiving, at the data processing device, an outcome of an objective refraction measurement of the at least one eye of the patient performed preoperatively and an outcome of an objective refraction measurement of the at least one eye of the patient performed postoperatively, and determining, at the data processing device, an outcome of a postoperative subjective refraction measurement of the at least one eye of the patient based on the outcome of the subjective refraction measurement of the at least one eye of the patient performed preoperatively, the outcome of the objective refraction measurement of the at least one eye of the patient performed preoperatively, and the outcome of the objective measurement of the at least one eye of the patient performed postoperatively.

### CROSS REFERENCE TO RELATED APPLICATIONS

This application is a national stage application of international patent application PCT/EP2022/083707 filed on Nov. 29, 2022, designating the U.S. and claiming priority from German patent application 10 2021 213 511.0, filed on Nov. 30, 2021, and the entire content of all applications is incorporated herein by reference.

### TECHNICAL FIELD

The present disclosure relates to the technical field of refractive eye surgery.

### BACKGROUND

In recent years, correcting a refractive error by way of refractive procedures on the eye of a patient has become ever more important, complementing the conventional correction of the refractive error using a pair of spectacles or contact lenses for example.

The umbrella term refractive surgery or refractive procedure subsumes eye operations which modify an overall refractive power of an eye of a patient and thus can replace conventional optical corrections such as spectacles or contact lenses, or are at least intended to significantly reduce the required strength thereof.

A refraction measurement can be performed before and after the refractive procedure. A refractive property of at least one (human) eye is determined within the scope of such a refraction determination or refraction measurement. In terms of refraction measurement, a distinction can be made between a subjective refraction measurement and an objective refraction measurement.

Methods and devices for objective refraction determination require no feedback from the person to be examined in relation to their visual perception. The refractive errors are measured, with computational preparation where necessary, from the directly measured variables. WO 2004/112576 A2 describes a method and a device for determining a measure of visual acuity on the basis of a measured wavefront aberration. Here, the measure for visual acuity is calculated on the basis of the measured wavefront aberration of lower and higher order using a point spread function. The objective refraction determination can be carried out with the aid of autorefractors, photorefractors, wavefront analyzers, retinoscopes, etc.

Methods for subjective refraction measurement are based on (subjective) feedback from a person to be examined in relation to their visual perception. One example for a subjective refraction measurement is a determination of the refractive properties on the basis of eye charts with ever decreasing optotypes (e.g., numbers or letters) or ever decreasing symbols, wherein the person to be examined provides feedback regarding the size down to which they can discern optotypes or symbols.

The subjective refraction determination can be implemented by means of a trial frame and trial lenses or a manual or digital phoropter and using optotypes, which are displayed on an external monitor. Here, a person to be examined observes the optotypes and an optician or ophthalmologist inserts different trial lenses with different corrective powers into the trial frame, or changes a correction setting if a phoropter is used. The person to be examined then provides feedback regarding which trial lenses or which settings of the phoropter allow the optotypes to be recognized to the best possible extent.

Here, the trial lenses used in the process or the corresponding correction settings of the phoropter are in each case assigned to a certain refractive error, which is corrected by the respective trial lens or the setting of the phoropter. Here, the subjective refraction determination explained above can be implemented separately for each eye (in monocular fashion) or else for both eyes together (in binocular fashion).

In the case of refractive procedures, the subjective refraction measurement can be implemented both before and after the procedure. In this case, measuring a postoperative subjective refraction can be part of an assessment regarding the success of a refractive procedure on the eye, comprising an assessment of patient satisfaction with the refractive procedure. In this context, the subjective refraction measurement can be determined both preoperatively (i.e. before the refractive procedure) and postoperatively (i.e. after the refractive procedure) in order to assess a visual function of a treated eye of the patient and a need for a further correction.

An outcome of the postoperative subjective refraction measurement can also be used for developing a suitable correction of a nomogram of a laser used during the respective procedure in order to improve future refractive procedures using a laser, optionally the laser used during the refractive procedure.

Since the outcome of the subjective refraction measurement is also based on a subjective perception of the respective patient, reproducibility of the outcome of the subjective refraction measurement can be challenging in comparison with the objective refraction measurement, inter alia on account of varying cooperativeness of the respective patient and their expectations regarding the outcome of the refractive procedure.

Thus, removing the need for performing a postoperative subjective refraction measurement would be advantageous both for postoperative care and for the nomogram correction.

Various approaches for estimating an outcome of a postoperative subjective refraction measurement on the basis of an outcome of a postoperative objective refraction measurement of the eye (e.g. using an autorefractor, a photorefractor, a wavefront analyzer and/or a retinoscope; see above) exist, which can reproducibly determine the refractive properties of the eye.

EP3321831A1 describes a device for ascertaining predicted subjective refraction data or predicted subjective correction values of an eye to be examined on the basis of objective refraction data of the eye to be examined. The device comprises an evaluation apparatus with a calculation unit, which ascertains the predicted subjective refraction data or predicted subjective correction values of the eye from the objective refraction data of the eye by means of a function. The function is a nonlinear multidimensional function or a family of nonlinear multidimensional functions, which is the outcome of training a regression model or classification model, wherein the regression model or classification model has been trained on the basis of a training data record, which, for a multiplicity of subjects, in each case comprises at least objective refraction data and assigned ascertained subjective refraction data or assigned ascertained subjective correction values.

A disadvantage of the methods and devices described in the prior art is that these might not consider a cortical adaptation of the patient when ascertaining the postoperative subjective refraction measurement; however, this is also taken into account during the conventional or actually performed postoperative subjective refraction measurement.

The cortical adaptation, sometimes also referred to as refractive adaptation, can be defined as a gradual adaptation that occurs in the visual cortex, which is part of the cerebral cortex in the brain of the patient, when a refractive error is present in one or both eyes of the patient. In more detail, the brain of the patient automatically adjusts to a refractive error in one or both eyes of the patient to a certain degree, in order to compensate the refractive error. Thus, what may arise is that the eye or eyes of the patient have a refractive error corresponding to a specific value when the latter is measured objectively, but the refractive error may be perceived as a different value, possibly or typically closer to the normal value, when the same person is tested using subjective methods. The reason for this discrepancy between the two values can be found in the fact that the brain of the patient learns over time to compensate the refractive error in the eye or eyes of the patient to a certain degree in order to perceive the surroundings more normally.

The subjective refraction measurement may also be referred to as manifest refraction measurement or subjective manifest refraction measurement. These subjective measurements may comprise the measurement of a refractive error in an eye of a patient, wherein for example various lenses are placed in front of the eye of the patient in the process and the patient is requested to describe whether their vision is better or worse with the current lens in comparison with the previous lens.

### SUMMARY

Provided is a method, the method comprising receiving, at a data processing device, an outcome of a subjective refraction measurement of at least one eye of a patient performed preoperatively, receiving, at the data processing device, an outcome of an objective refraction measurement of the at least one eye of the patient performed preoperatively and postoperatively, and determining, at the data processing device, an outcome of a postoperative subjective refraction measurement of the at least one eye of the patient at least on the basis of the outcome of the subjective refraction measurement of the at least one eye of the patient performed preoperatively and the outcome of the objective refraction measurement of the at least one eye of the patient performed preoperatively and postoperatively.

Provided is a computing device, wherein the computing device comprises means being implemented in software and/or hardware for determining an outcome of a postoperative subjective refraction measurement of at least one eye of a patient at least on the basis of an outcome of a subjective refraction measurement of the at least one eye of the patient performed preoperatively and an outcome of an objective refraction measurement of the at least one eye of the patient performed preoperatively and postoperatively.

Provided is a computer program product, the computer program product comprising commands which, when the program is executed by a computer, cause the latter to carry out the method described above at least in part.

Provided is a method, the method comprising providing an artificial intelligence-based model at a data processing device, and training, at the data processing device, the artificial intelligence-based model such that the model post training is configured to carry out the method described above at least in part.

A method for determining an outcome of a postoperative subjective refraction measurement of at least one eye of a patient is provided. The outcome of the postoperative subjective refraction measurement may be determined or predicted at least on the basis of an outcome of a subjective refraction measurement of the at least one eye of the patient performed preoperatively.

The method can effectively replace an implementation of a subjective refraction measurement following a refractive surgical procedure on at least one eye of the patient which has modified a refractive power of the at least one eye. Thus, the method can be used to predict an outcome of a subjective refraction measurement following the procedure.

Optionally, this can be achieved by determining the expected outcome, i.e. the outcome of an actually performed subjective refraction measurement after the procedure (i.e. postoperatively), at least inter alia on the basis of already known data from a subjective refraction measurement performed before the procedure (i.e. preoperatively).

Therefore, a cortical adaptation of the patient can be considered when determining the (expected) outcome of the postoperative subjective refraction measurement based on the data obtained by the subjective refraction measurement performed preoperatively, i.e. the data influenced by a subjective perception of the respective patient. The conventional methods for determining the postoperative subjective refraction measurement, which resort exclusively to data obtained by objective refraction measurements, do not take account of the cortical adaptation. As a result, an improved, especially more accurate, determination of the (expected) outcome of the postoperative subjective refraction measurement can be obtained by the method described herein.

The at least one eye of the patient can be the eye to be treated or the treated eye. The refractive procedure may comprise an ablation of tissue from the at least one eye by means of a laser, for example an excimer laser, a femtosecond laser, and/or a solid-state laser. The refractive procedure can be a laser in-situ keratomileusis (LASIK) or a photorefractive keratectomy (PRK). The refractive procedure can comprise, inter alia, an insertion of an intraocular lens.

However, to determine the outcome of the postoperative subjective refraction measurement, the method is not limited to using the outcome of the subjective preoperative refraction measurement.

Optionally, the outcome of the postoperative subjective refraction measurement can be determined on the basis of an outcome of an objective refraction measurement of the at least one eye of the patient performed preoperatively and/or postoperatively.

That is to say, in addition to the subjective data, outcomes or data for determining the (expected) outcome of the postoperative subjective refraction measurement obtained through objective refraction measurement can be used, for example, to further improve an accuracy of the determination.

The outcome of the postoperative subjective refraction measurement can be determined by means of a model, optionally a model based on artificial intelligence. As input data, the model can receive the outcome of the subjective refraction measurement performed preoperatively and the outcome of the objective refraction measurement performed preoperatively and/or postoperatively. The model can determine the outcome of the postoperative subjective refraction measurement on the basis of the input data.

In the present case, a model can be understood to be an algorithm, for example a deterministic algorithm, which models or describes a relationship between the input data, which can also be referred to as input parameters, and the output data, in this case the outcome of the postoperative subjective refraction measurement to be determined. By means of the relationship of input data to output data known to the algorithm, the algorithm may be configured to determine, more particularly calculate, the outcome of the postoperative subjective refraction measurement. The algorithm or a model may be implemented in soft- and/or hardware.

A model based on artificial intelligence may comprise an artificial neural network, optionally a feedforward network with one or more layers and/or a recurrent network. The artificial neural network, which may be or comprise a convolutional neural network (CNN), may be configured or trained to model the relationship between the input data and the output data, in this case the outcome of the postoperative subjective refraction measurement, as described above. Consequently, the artificial neural network can determine the outcome of the postoperative subjective refraction measurement on the basis of the input data, which at least comprise the outcome of the preoperative subjective refraction measurement in this case.

The artificial neural network can receive as input data both the outcome of the subjective refraction measurement performed preoperatively and the outcome of the objective refraction measurement performed preoperatively and postoperatively.

The artificial neural network can learn and model a relationship between the outcomes of the preoperative and postoperative and objective and subjective refraction measurements. Learning can be part of the above-described method.

Consequently, either in combination with or independently of the above-described method, the disclosure also relates to a method for training an artificial intelligence-based model configured to determine the outcome of the postoperative subjective refraction measurement on the basis of the outcome of the subjective refraction measurement performed preoperatively and optionally also on the basis of the outcome of the objective refraction measurement performed preoperatively and/or postoperatively.

Training data optionally containing a plurality of training data records, optionally from a plurality of patients, can be used to learn the relationship(s).

Consequently, either in combination with or independently of the above-described methods, the disclosure also relates to a training data record for training an artificial intelligence-based model configured to determine the outcome of the postoperative subjective refraction measurement on the basis of the outcome of the subjective refraction measurement performed preoperatively and optionally also on the basis of the outcome of the objective refraction measurement performed preoperatively and/or postoperatively.

The training data records may each contain an outcome of a preoperative subjective and objective refraction measurement and postoperative subjective and objective refraction measurement. The training data records may originate from actually performed refractive procedures.

The model might have a patient-specific model which determines a first preliminary outcome of the postoperative subjective refraction measurement on the basis of the outcome of the subjective refraction measurement performed preoperatively and the outcome of the objective refraction measurement performed preoperatively and/or postoperatively and optionally on the basis of patient information.

Consequently, either in combination with or independently of the above-described methods and the above-described training data record, the disclosure also relates to a model configured to determine the outcome of the postoperative subjective refraction measurement on the basis of the outcome of the subjective refraction measurement performed preoperatively and optionally also on the basis of the outcome of the objective refraction measurement performed preoperatively and/or postoperatively.

The patient-specific model can be based on artificial intelligence and trained using a training data record comprising outcomes of a plurality of objective refraction measurements performed preoperatively and/or postoperatively and outcomes of a plurality of subjective refraction measurements, corresponding to the latter, performed preoperatively and postoperatively. This may relate to the above-described training data record.

The training data record used to train the artificial intelligence-based patient-specific model may also comprise patient information corresponding to the outcomes of the plurality of objective refraction measurements performed preoperatively and/or postoperatively and to the outcomes of the plurality of subjective refraction measurements, corresponding to the latter, performed preoperatively and postoperatively.

The patient information may comprise information regarding an eye biometry of the at least one eye of the patient, an age of the patient, and/or a sex of the patient. The information regarding an eye biometry of the at least one eye of the patient may comprise an axial length of the at least one eye of the patient, a curvature of an anterior corneal surface of the at least one eye of the patient, an anterior chamber depth of the at least one eye of the patient, the horizontal visible iris diameter (white-to-white diameter, WTW), a wavefront aberrometry and/or an anterior segment biometry, in which only the front third of the at least one eye of the patient is measured.

Accordingly, it is possible to consider factors for estimating the cortical adaptation of the subject or patient which are patient specific and may contribute to translating objective data to subjective data, for example the age, the sex and/or outcomes of further postoperative and/or preoperative measurements, in particular on the anterior eye segment.

It is conceivable that the method for determining the outcome of the postoperative subjective refraction measurement also includes training the patient-specific model with the training data record. The patient-specific model can be trained before the patient-specific model determines the first preliminary outcome of the postoperative subjective refraction measurement, i.e. before the model is actually used. In addition to that or in an alternative, the patient-specific model can be trained after or while the patient-specific model determines the first preliminary outcome of the postoperative subjective refraction measurement, i.e. while and after the model is actually used.

Training may comprise learning a relationship that describes how a difference between the outcome of the preoperative objective refraction measurement and the postoperative objective refraction measurement or how a magnitude/size of a change in this outcome affects a change of the subjective refraction measurement from pre-operation to post-operation. Accordingly, it is possible to learn how an (objectively measurable) change in refractive power of the treated eye caused by the refractive procedure affects the outcome of the postoperative subjective refraction measurement when the outcome of the preoperative subjective refraction measurement is taken into account or used as a starting point. This part of the model, i.e. the above-described so-called patient-specific model, relates to the refractive procedure itself and in each case assigns the change in the outcome between preoperative and postoperative objective refraction measurement to the outcome of the preoperative and postoperative subjective refraction measurement. Written as a formula, this may be represented as follows:

$${postMR}_{1} = preMR ⊖ ( preObj ⊖ postObj ) , \qquad ( 1 )$$

where postMR1 is the first preliminary outcome of the postoperative subjective refraction measurement, preMR is the outcome of the subjective refraction measurement performed preoperatively, preObj is the outcome of the objective refraction measurement performed preoperatively, postObj is the outcome of the objective refraction measurement performed postoperatively (the aforementioned parameters/pieces of information can be part of the training data) and ⊖ is an operator to be learned.

This equation (1) might essentially represent the patient-specific model which determines the first preliminary outcome of the postoperative subjective refraction measurement on the basis of the outcome of the subjective refraction measurement performed preoperatively and the outcome of the objective refraction measurement performed preoperatively and/or postoperatively.

Equation (1) only represents a specific example of several possible implementations of the patient-specific model, and in no way is the disclosure limited thereto.

In addition or as an alternative to the patient-specific model, the model may have a cortical adaptation model which determines a second preliminary outcome of the postoperative subjective refraction measurement on the basis of the outcome of the objective refraction measurement performed postoperatively.

The cortical adaptation model can be based on artificial intelligence and trained using a training data record comprising outcomes of a plurality of objective refraction measurements performed preoperatively and outcomes of a plurality of subjective refraction measurements, corresponding to the latter, performed preoperatively.

It is conceivable that the method also includes training the cortical adaptation model using the training data record, which may be the same training data record or a different training data record to any of the above-described training data records. The cortical adaptation model can be trained before the cortical adaptation model determines the second preliminary outcome of the postoperative subjective refraction measurement, i.e. before the model is actually used. In addition to that or in an alternative, the cortical adaptation model can be trained after or while the patient-specific model determines the first preliminary outcome of the postoperative subjective refraction measurement, i.e. while and after the model is actually used.

Training the cortical adaptation model may comprise learning a relationship between the outcomes of the preoperative subjective refraction measurements and the preoperative objective refraction measurements. This serves to learn a cortical adaptation and, written as a formula, can be represented as follows:

$$preMR = a ⊗ preObj ⊕ b , \qquad ( 2 )$$

where in this case, too, preMR is the outcome of the subjective refraction measurement performed preoperatively and preObj is the outcome of the objective refraction measurement performed preoperatively (the aforementioned parameters/pieces of information are part of the training data), ⊗ and ⊕ each are operators to be learned and a and b each are parameters to be learned.

The training steps described in equations (1) and (2) can be performed simultaneously. Thus, all subjective and objective preoperative and postoperative measurements of the training data record can be used to train the patient-specific model and the cortical adaptation model.

Equation (3), below, may comprise the parameters a and b, learned by means of equation (2), and the operator ⊗ and may essentially represent the above-described cortical adaptation model which determines a second preliminary outcome of the postoperative subjective refraction measurement on the basis of the outcome of the objective refraction measurement performed postoperatively. That is to say, in order to obtain a second estimator of the outcome of the postoperative subjective refraction measurement (to be expected or determined), the parameters a and b determined by means of equation (2) described above can be used together with the operator ⊗, and a second estimator for the outcome of the postoperative subjective refraction measurement (to be expected or determined) is derived with the aid thereof on the basis of the postoperative objective refraction measurement. Written as a formula, this may be represented as follows:

$${postMR}_{2} = a ⊗ postObj ⊕ b , \qquad ( 3 )$$

where postMR2 is the second preliminary outcome of the postoperative subjective refraction measurement, postObj is the outcome of the objective refraction measurement performed postoperatively, a and b are the parameters learned by means of equation (2) and ⊗ is the operator learned by means of equation (2).

Equations (2) and (3) only represent a specific example of several possible implementations of the cortical adaptation model, and in no way is the disclosure limited thereto.

The model may include a combination model which determines the outcome of the postoperative subjective refraction measurement on the basis of the first and second preliminary outcome of the postoperative subjective refraction measurement.

In other words, the combination model can obtain as input data the two preliminary outcomes of the postoperative subjective refraction measurement, optionally determined as described above, from the patient-specific model and the adaptation model and can on the basis thereof determine the outcome of the postoperative subjective refraction measurement to be determined by means of the method.

That is to say, the determination of the two above-described preliminary outcomes by means of the cortical adaptation model and patient-specific model can be followed by the first and the second estimator being combined; written as a formula, this may be represented as follows:

$$postMR = α ⊗ {postMR}_{1} ⊕ β ⊗ {postMR}_{2} , \qquad ( 4 )$$

where postMR1 is the first preliminary outcome of the postoperative subjective refraction measurement, postMR2 is the second preliminary outcome of the postoperative subjective refraction measurement and postMR is the outcome of the postoperative subjective refraction measurement to be determined by means of the method, ⊗ and ⊕ each are operators learned using equation (2), and a and B each are parameters to be learned.

Training the combination model can also be a part, optionally the only part, of the above-described training method. Training can be implemented before the combination model determines the first outcome of the postoperative subjective refraction measurement, i.e. before the model is actually used. In addition to that or in an alternative, the combination model can be trained after or while the combination model determines the first preliminary outcome of the postoperative subjective refraction measurement, i.e. while and after the model is actually used.

Equation (4) only represents a specific example of several possible implementations of the combination model, and in no way is the disclosure limited thereto.

It is conceivable that the above-described training of the cortical adaptation model, patient-specific model and/or combination model is implemented on an individual basis or together.

One or more algorithms from the field of machine learning can be used within the scope of the training. This is advantageous because the artificially intelligent model thus learns from examples, i.e. the above-described training data, and is capable of generalizing these following the completion of the learning phase. That is to say, the algorithm or algorithms can construct a statistical model based on the training data during the training or machine learning steps. In the process, examples are not simply learned by rote; instead, patterns and rules, i.e. the above-described relationships/operators and parameters, are identified in the training data. Thus, following the training, the model is also capable of assessing unknown data (so-called learning transfer) and thus is suitable for determining the outcome of the postoperative subjective refraction measurement in the manner described above.

In more complex nonlinear machine learning models, the relationships, i.e. the ⊗, ⊕, ⊖ operators, can advantageously be estimated or determined nonlinearly since there are complex interactions between a plurality of parameters in that case. In addition to that or alternatively, the combination model, which establishes a relationship between the output of the cortical adaptation model and of the patient-specific model, can advantageously be learnt in an end-to-end model.

The above-described renders it possible to assess the quality of a vision correction achieved by the refractive procedure without this needing a postoperative subjective refraction measurement.

It is conceivable that the method comprises a correction of a nomogram of a laser, optionally used for the refractive procedure, on the basis of the (determined) outcome of the postoperative subjective refraction measurement.

A nomogram should be understood to be a mathematical model, optionally a computer-implemented model, which correlates the refraction correction obtained by a laser correction procedure with certain input laser settings. The laser settings can relate to the extent of the correction intended to be implemented at each eye or at an individual eye of the patient by the laser correction equipment. For example, surgeons can use such a nomogram in order to find the correct laser settings for each eye or for an individual eye of the patient; these settings are input into the laser correction equipment as inputs, depending on the correction intended to be achieved by the surgeon.

Additionally, or alternatively, a device configured to carry out the method at least in part can be provided. The device can be or comprise a computing device, optionally a computer, which may be part of a laser system configured to carry out a refractive procedure. In addition to that or in an alternative, the laser system itself, configured to carry out the method at least in part, can be provided. Further, in addition to that or in an alternative, a computer program product comprising commands which, when the program is executed by a computer, cause the latter to carry out the method at least in part can be provided. Further, a training data record can be provided and can be trained by an artificial intelligence-based model such that the latter is capable of determining an outcome of a postoperative subjective refraction measurement by means of the method. It is also possible to provide a method for training an artificial intelligence-based model or a training method for an artificial intelligence-based model, in which the model is a trained, optionally using the above-described training data record, such that the above-described model post training is configured to carry out the above-described method at least in part.

What was described above in relation to the method also applies analogously to the device, the training method, the computer program product, and the training data record.

The features and examples specified above and explained below should not only be considered to be disclosed in the respective explicitly mentioned combinations, but are also comprised by the disclosure in other technically advantageous combinations and embodiments.

### BRIEF DESCRIPTION OF THE DRAWINGS

Optional details and advantages of the disclosure should now be explained in more detail on the basis of the following examples with reference being made to the FIGS., in which:

FIG. 1 shows a flowchart for explaining a method for determining an outcome of a postoperative subjective refraction measurement,

FIG. 2 shows a further flowchart for the detailed explanation of a method for training an artificial intelligence-based model which is used in the method depicted in FIG. 1 for determining the outcome of the postoperative subjective refraction measurement, and

FIG. 3 shows a further flowchart for the detailed explanation of a method for determining the outcome of the postoperative subjective refraction measurement using the model trained in accordance with the method from FIG. 2.

### DESCRIPTION

The same or similar elements are denoted by the same reference symbols in the following description for reasons of simplicity. Neither the content of the FIGS. nor the following description thereof should in any way be construed as limiting.

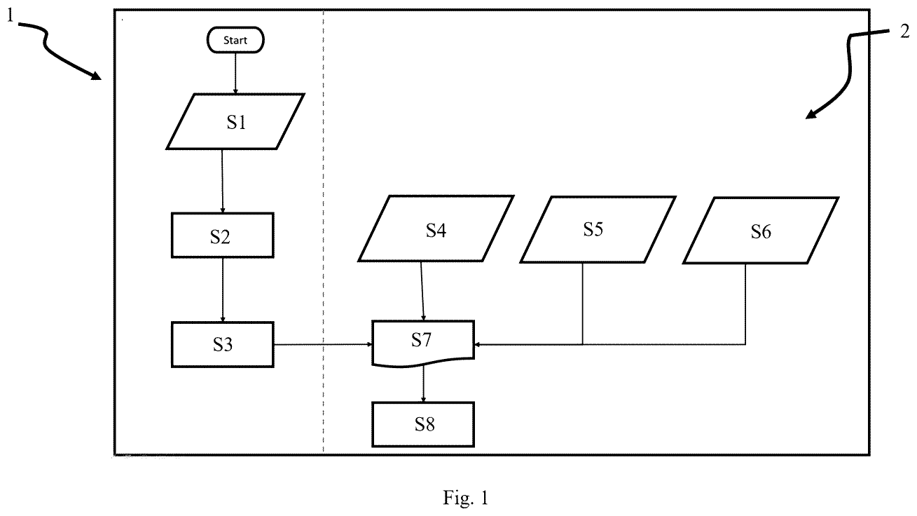

As may be gathered from FIG. 1, the method for determining the outcome of the postoperative subjective refraction measurement postMR can essentially be subdivided into two blocks or partial methods 1, 2.

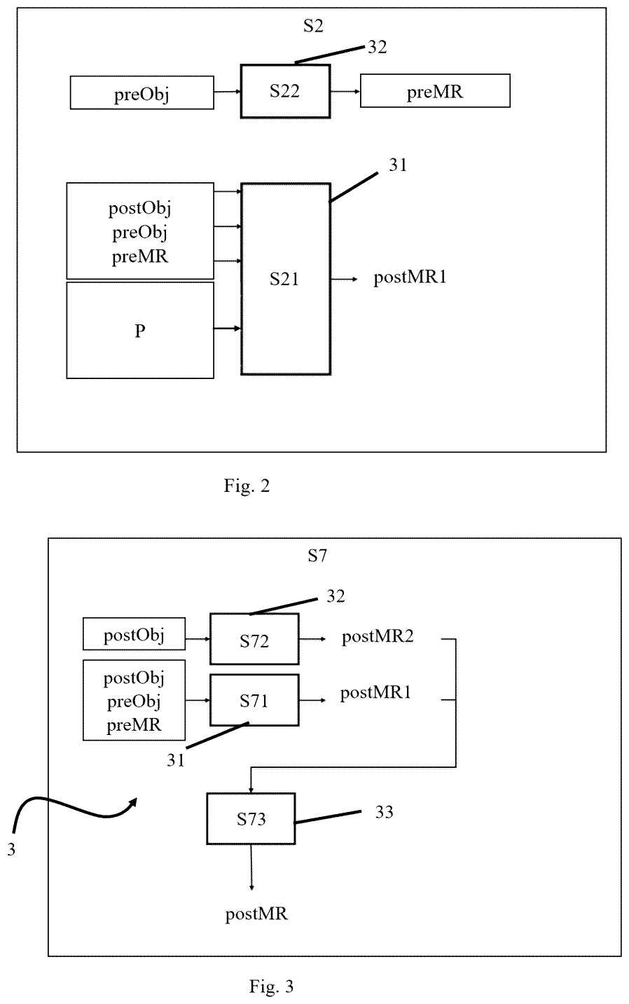

A first of the two blocks 1 comprises a training method, as depicted on the left in FIG. 1 and, in part, in detail in FIG. 2, with essentially three steps S1, S2, S3.

A second of the two blocks 2, which follows the first block 1 or in the present case is implemented after the first block 1, comprises the determination of the outcome of the postoperative subjective refraction measurement, which in the present case is implemented in essentially five steps S4, S5, S6, S7, S8 and is depicted to the right in FIG. 1 and, in part, in detail in FIG. 3.

Below, the determination of the outcome of the postoperative subjective refraction measurement postMR for at least one eye of a patient, i.e. the second block 2 depicted to the right in FIG. 1, is described first.

Determining the outcome of the postoperative subjective refraction measurement postMR initially includes a retrieval of input data, which is presently implemented in three steps S4, S5, S6 running in parallel. However, the input data can also be retrieved successively or in a single step.

The input data comprise an outcome of a subjective refraction measurement preMR performed preoperatively on a patient and an outcome of an objective refraction measurement preObj performed preoperatively on the patient; these outcomes are retrieved together in a first step S4 of determining the outcome of the postoperative subjective refraction measurement postMR. The patient is the person who undergoes the refractive procedure on at least one of their eyes. Both the subjective refraction measurement preMR performed preoperatively and the objective refraction measurement preObj performed preoperatively were at least performed on the at least one eye (to be treated) of the patient.

The input data also comprise an outcome of an objective refraction measurement postObj performed postoperatively on the patient; this outcome is retrieved in a second step S5 of determining the outcome of the postoperative subjective refraction measurement postMR. The objective refraction measurement postMR performed postoperatively was also at least performed on the at least one eye (to be treated) of the patient.

The input data also comprise patient information P (shown on FIG. 2) which may also be referred to as patient-specific information of the patient to be treated or of the treated patient and which can be retrieved in a third step S6 of determining the outcome of the postoperative subjective refraction measurement postMR.

The patient information P can include an age of the patient, a sex of the patient, and/or an eye biometry of the at least one eye (to be treated and/or already treated) of the patient.

The retrieval may comprise accessing (a so-called “read operation”) a memory (not depicted here), for example a buffer memory, of a computing device. The input data may be stored in this memory. In addition to that or in an alternative, it is also conceivable that the input data are at least in part stored in a cloud and retrieved from this cloud.

In a specific example, in which the outcome of the postoperative subjective refraction measurement postMR is determined after the refractive procedure, the outcome of the subjective refraction measurement preMR performed preoperatively and the outcome of the objective refraction measurement preObj performed preoperatively can be retrieved in the first step S4 and the patient information P can be retrieved in the third step S6 from one or more clouds, whereas the outcome of the objective refraction measurement postObj performed postoperatively can be retrieved in the second step S5 from a local memory in a computing device which performs the method for determining the outcome of the postoperative subjective refraction measurement postMR at least in part.

In a fourth step S7, which follows or builds on the first three steps S4, S5, S6, the outcome of the postoperative subjective refraction measurement postMR is determined in the actual sense.

To this end, the previously retrieved input data (steps S4, S5, S6; see above) are processed in the fourth step S7 by a model 3, which is based on artificial intelligence in the present case and which is depicted in detail in FIG. 3, in order thus to use the determined outcome of the postoperative subjective refraction measurement postMR for a nomogram correction in a fifth step S8.

The outcome of the postoperative subjective refraction measurement postMR is determined, for example by the above-described computing device capable of retrieving the input data, in the fourth step S7 on the basis of the outcome of the objective refraction measurement preObj, postObj performed preoperatively and postoperatively, the patient information P and the outcome of the subjective refraction measurement preMR performed preoperatively. This fourth step S7 and the model 3 based on artificial intelligence are depicted in detail in FIG. 3 and are described below with reference to FIG. 3.

The model 3 based on artificial intelligence includes a patient-specific model 31, a cortical adaptation model 32, and a combination model 33, all of which are based on artificial intelligence in the present case. However, it is also conceivable that only one, two or none of the three models 31, 32, 33 are based on artificial intelligence.

In a first partial step S71, the patient-specific model 31 determines a first preliminary outcome of the postoperative subjective refraction measurement postMR1 on the basis of the outcome of the subjective refraction measurement preMR performed preoperatively and the outcome of the objective refraction measurement preObj, postObj performed preoperatively and postoperatively and on the basis of the patient information P.

In a second partial step S72, the cortical adaptation model 32 determines a second preliminary outcome of the postoperative subjective refraction measurement postMR2 on the basis of the outcome of the objective refraction measurement postObj performed postoperatively.

In a third partial step S73, the combination model 33 determines the outcome of the postoperative subjective refraction measurement postMR on the basis of the first and second preliminary outcome of the postoperative subjective refraction measurement postMR1, postMR2.

In step S8 of the method which follows the determination of the postoperative subjective refraction measurement postMR, a nomogram of a laser, presently used for the refractive procedure, is corrected on the basis of the outcome of the postoperative subjective refraction measurement postMR determined in the fourth step S7.

Since a model 3 based on artificial intelligence is used in the present example as the model for predicting or determining the outcome of the postoperative subjective refraction measurement postMR, the method comprises the preceding training method which is depicted in the left part of FIG. 1 and, in part, in detail in FIG. 2, as indicated at the outset. This training method or method for training the model 3 is described in detail below with reference to FIG. 1, especially the left part of FIG. 1, and FIG. 2.

In the present case, the training method is performed prior to the above-described determination of the outcome of the postoperative subjective refraction measurement postMR and starts with a first step S1, in which training data are retrieved or read. Here, too, the retrieval may comprise accessing (a so-called “read operation”) a memory (not depicted here), for example a buffer memory, of a computing device. The training data may be stored in this memory. In addition to that or in an alternative, it is also conceivable that the training data are at least in part retrieved from a cloud.

The training data comprise a plurality of training data records, each of which originate from refractive procedures performed in the past on different patients. The refractive procedure can be the same refractive procedure or a different refractive procedure to the one for which the model 3 to be trained is used post training.

Each of the training data records comprises outcomes of an objective refraction measurement preObj, postObj performed preoperatively and postoperatively, in each case originating from a single refractive procedure, optionally patient information regarding the patient, and an outcome of a subjective refraction measurement preMR, postMR performed preoperatively and postoperatively.

The training data retrieved in the first step S1 are used in a second step S2 of the training method, in each case to train the patient-specific model 31 and the cortical adaptation model 32.

More precisely, the patient-specific model is trained in a first partial step S21 of the second step S2 of the training method using the outcomes of the plurality of objective refraction measurements preObj, postObj performed preoperatively and postoperatively and contained in the training data, using the outcomes of the plurality of subjective refraction measurements preMR, postMR, corresponding thereto, performed preoperatively and postoperatively and contained in the training data, and the respective patient information P.

The cortical adaptation model 32 is trained in a second partial step S22 of the second step S2 of the training method using the outcomes of the plurality of objective refraction measurements preObj performed preoperatively and contained in the training data and using the outcomes of the plurality of subjective refraction measurements preMR, corresponding thereto, performed preoperatively and contained in the training data.

At least parts of the first and the second partial steps S21 and S22 of the second step S2 of the training method can be implemented in parallel or simultaneously, or successively.

Since the model 3 trained thus, in particular the cortical adaptation model 32, has learned a relationship between outcomes of objective and subjective refraction measurements, it is possible to give consideration to a cortical adaptation when determining or predicting the outcomes of the subjective refraction measurements postMR performed or to be performed postoperatively. This represents an advantage over the conventional methods which predict or determine the outcome of the subjective refraction measurements performed or to be performed postoperatively merely on the basis of outcomes of objective data.

In a third and presently last step S3 of the training method, the trained model is made available to the computing device which, as described above, carries out the second block 2 (steps S4, S5, S6, S7, S8; see above) of the method using the trained model.

Moreover, it is possible to use methods such as ray tracing to create an optical model of an eye which is as realistic as possible. This optical model itself could be a further input, similar to age, sex, etc., in order to train or use the artificial intelligence-based model as described above.

### REFERENCE SYMBOLS

1 First part of the method for determining the outcome of the postoperative subjective refraction measurement or of the training method
2 Second part of the method for determining the outcome of the postoperative subjective refraction measurement, or determining the outcome of the postoperative subjective refraction measurement using the trained (prediction) model
3 (Prediction) model
31 Patient-specific model
32 Cortical adaptation model
33 Combination model
S1-S7 Steps of the method for determining the outcome of the postoperative subjective refraction measurement
P Patient information
postMR Outcome of the postoperative subjective refraction measurement
postMR1 First preliminary outcome of the postoperative subjective refraction measurement
postMR2 Second preliminary outcome of the postoperative subjective refraction measurement
postObj Outcome of the postoperative objective refraction measurement
preMR Outcome of the preoperative subjective refraction measurement
preObj Outcome of the preoperative objective refraction measurement

What is claimed is:

1-16. (canceled)

17. A method, comprising:
receiving, at a data processing device, an outcome of a subjective refraction measurement of at least one eye of a patient performed preoperatively,
receiving, at the data processing device, an outcome of an objective refraction measurement of the at least one eye of the patient performed preoperatively and an outcome of an objective refraction measurement of the at least one eye of the patient performed postoperatively, and
determining, at the data processing device, an outcome of a postoperative subjective refraction measurement of the at least one eye of the patient at least based on the outcome of the subjective refraction measurement of the at least one eye of the patient performed preoperatively, the outcome of the objective refraction measurement of the at least one eye of the patient performed preoperatively, and the outcome of the objective measurement of the at least one eye of the patient performed postoperatively.

18. The method according to claim 17, wherein the data processing device comprises a model, the method further comprising:
receiving, as input data, the outcome of the subjective refraction measurement performed preoperatively, the outcome of the objective refraction measurement performed preoperatively, and the outcome of the objective refraction measurement performed postoperatively at the model, and
determining, at the data processing device using the model, the outcome of the postoperative subjective refraction measurement based on the input data.

19. The method according to claim 18, wherein the model is based on artificial intelligence.

20. The method according to claim 18, wherein the model includes a patient-specific model, the method further comprising:
determining, at the data processing device using the patient-specific model, a first preliminary outcome of the postoperative subjective refraction measurement based on the outcome of the subjective refraction measurement performed preoperatively, the outcome of the objective refraction measurement performed preoperatively, and the outcome of the objective refraction measurement performed postoperatively.

21. The method according to claim 20, comprising:
determining, at the data processing device using the patient-specific model, the first preliminary outcome of the postoperative subjective refraction measurement additionally based on patient information.

22. The method according to claim 20, wherein the patient-specific model is based on artificial intelligence and trained using a training data record comprising outcomes of a plurality of objective refraction measurements performed preoperatively, outcomes of a plurality of objective refraction measurements performed postoperatively, outcomes of a plurality of subjective refraction measurements performed preoperatively, and outcomes of a plurality of subjective refraction measurements performed postoperatively, the respective outcomes corresponding to each other.

23. The method according to claim 22, wherein the training data record used to train the artificial intelligence-based patient-specific model comprises patient information corresponding to the outcomes of the plurality of objective refraction measurements performed preoperatively, the outcomes of the plurality of objective refraction measurements performed postoperatively, outcomes of a plurality of subjective refraction measurements performed preoperatively, and outcomes of a plurality of subjective refraction measurements performed postoperatively.

24. The method according to claim 23, wherein the patient information comprises an eye biometry of the at least one eye of the patient, an age of the patient, and/or a sex of the patient.

25. The method according to claim 18, wherein the model includes a cortical adaptation model, the method comprising:
determining, at the data processing device using the cortical adaptation model, a second preliminary outcome of the postoperative subjective refraction measurement based on the outcome of the objective refraction measurement performed postoperatively.

26. The method according to claim 25, wherein the cortical adaptation model is based on artificial intelligence and trained using a training data record comprising outcomes of a plurality of objective refraction measurements performed preoperatively and outcomes of a plurality of subjective refraction measurements performed preoperatively, the respective outcomes corresponding to each other.

27. The method according to claim 18, wherein the model includes a patient-specific model, a cortical adaptation model, and a combination model, the method comprising:
determining, at the data processing device using the patient-specific model, a first preliminary outcome of the postoperative subjective refraction measurement based on the outcome of the subjective refraction measurement performed preoperatively, the outcome of the objective refraction measurement performed preoperatively, and the outcome of the objective refraction measurement performed postoperatively,
determining, at the data processing device using the cortical adaptation model, a second preliminary outcome of the postoperative subjective refraction measurement based on the outcome of the objective refraction measurement performed postoperatively, and
determining, at the data processing device using the combination model, the outcome of the postoperative subjective refraction measurement based on the first and the second preliminary outcomes of the postoperative subjective refraction measurement.

28. The method according to claim 17, the method further comprising:
correcting a nomogram of a laser based on the outcome of the postoperative subjective refraction measurement.

29. The method as claimed in claim 28, the method comprising:
using the corrected nomogram in a refractive procedure.

30. A computing device, comprising:
means being implemented in software and/or hardware for:
receiving, at the computing device, an outcome of a subjective refraction measurement of at least one eye of a patient performed preoperatively,
receiving, at the computing device, an outcome of an objective refraction measurement of the at least one eye of the patient performed preoperatively and an outcome of an objective refraction measurement of the at least one eye of the patient performed postoperatively, and
determining, at the computing device, an outcome of a postoperative subjective refraction measurement of the at least one eye of the patient at least based on the outcome of the subjective refraction measurement of the at least one eye of the patient performed preoperatively, the outcome of the objective refraction measurement of the at least one eye of the patient performed preoperatively, and the outcome of the objective measurement of the at least one eye of the patient performed postoperatively

31. The computing device according to claim 30, wherein the computing device is part of a laser system being configured to perform a refractive procedure based on the determined outcome of the postoperative subjective refraction measurement.

32. A computer program product, the computer program product comprising commands which, when the program is executed by a computer, cause the computer to carry out the method according to claim 17.

33. A method, comprising:
providing an artificial intelligence-based model at a data processing device, and
training, at the data processing device, the artificial intelligence-based model such that the artificial intelligence-based model is configured to carry out the method according to claim 17 post training.
